(19) United States
(12) Patent Application Publication
(10) Pub. No.: US 2017/0007128 A1
(43) Pub. Date: Jan. 12, 2017

(54) OUTPUT DEVICE, OUTPUT METHOD, AND RECORDING MEDIUM

(71) Applicant: FUJITSU LIMITED, Kawasaki-shi (JP)

(72) Inventors: Kosei Takano, Fujimino (JP); Masayoshi Hoshiya, Yokohama (JP); Masatsugu Isogai, Yokohama (JP)

(21) Appl. No.: 15/199,011

(22) Filed: Jun. 30, 2016

(30) Foreign Application Priority Data
Jul. 7, 2015 (JP) 2015-136375

(51) Int. Cl. A61B 5/00; A61B 5/022; G06F 17/30; A61B 5/08; G06F 7/20; G06F 19/00; A61B 5/01

(52) U.S. Cl. CPC A61B 5/0022; G06F 19/3418; A61B 5/022; A61B 5/01; A61B 5/082; A61B 5/4815; G06F 7/20; G06F 17/30312

(57) ABSTRACT

An output device includes a processor, wherein the processor executes a process. The process includes acquiring information including user identification information, device identification information for identifying a measuring device, a measurement value of each measurement type obtained by the measuring device, and measurement time of the measurement value, from different measuring devices. The process includes identifying information including measurement values associated with the same user identification information and the same measurement type, from the acquired information. The process includes determining whether time-series variation in the measurement values included in the identified information is different between a first measuring device and a second measuring device identified by the device identification information included in the identified information. The process includes outputting a result of difference when the time-series variation in the measurement values is different between the first measuring device and the second measuring device.

### CROSS-REFERENCE TO RELATED APPLICATION

This application is based upon and claims the benefit of priority of the prior Japanese Patent Application No. 2015-136375, filed on Jul. 7, 2015, the entire contents of which are incorporated herein by reference.

### FIELD

The embodiments discussed herein are related to an output device, an output method, and a recording medium.

### BACKGROUND

Various measuring devices have been recently used in various environments to measure biological information on a user, for example (e.g., Japanese Laid-open Patent Publication No. 2011-133300). Based on the measurement results, the user's health is managed. Examples of the measuring devices include, but are not limited to, pulsimeters that measure a pulse, sphygmomanometers that measure blood pressure, etc. These measuring devices are manufactured by various manufacturers, and various models are produced by each manufacturer. In a case where a measuring device is a pulsimeter, for example, examples of the pulsimeter include, but are not limited to, a contact pulsimeter that measures a pulse in contact with the body of the user, a non-contact pulsimeter that measures a pulse without being in contact with the body of the user, etc.

In a case where measurement is performed by a measurement method different from that recommended by the measuring device, a case where a measurement environment is different from that recommended for the measuring device, or a case where the measuring device is in an abnormal state, for example, a measurement result obtained by the measurement and output by the measuring device may possibly be different from that to be originally output. Furthermore, in a case where different measuring devices measure a single subject, they may possibly output different measurement results. If the measurement result-output by the measuring device is incorrect, the incorrectness fails to be detected.

### SUMMARY

According to an aspect of the embodiments, an output device includes a processor, wherein the processor executes a process. The process includes: acquiring information including user identification information, device identification information for identifying a measuring device, a measurement value of each measurement type obtained by the measuring device, and measurement time of the measurement value, from different measuring devices; identifying information including measurement values associated with the same user identification information and the same measurement type, from the acquired information; determining whether time-series variation in the measurement values included in the identified information is different between a first measuring device and a second measuring device identified by the device identification information included in the identified information; and outputting a result of difference when the time-series variation in the measurement values is different between the first measuring device and the second measuring device.

The object and advantages of the invention will be realized and attained by means of the elements and combinations particularly pointed out in the claims.

It is to be understood that both the foregoing general description and the following detailed description are exemplary and explanatory and are not restrictive of the invention, as claimed.

### BRIEF DESCRIPTION OF DRAWINGS

FIG. 1 is an example diagram for explaining a measuring system according to an embodiment;

FIG. 2 is an example diagram for explaining a measuring device;

FIG. 3 is an example diagram for explaining a server device;

FIG. 4 is an example diagram for explaining a record configuration of a user DB;

FIG. 5 is an example diagram for explaining a record configuration of an information DB;

FIG. 6 is an example diagram for explaining a terminal device;

FIG. 7 is an example flowchart of a processing operation performed by the server device relating to first output processing;

FIG. 8 is an example flowchart of a processing operation performed by the server device relating to second output processing; and

FIG. 9 is an example diagram for explaining a computer that executes an output program.

### DESCRIPTION OF EMBODIMENTS

Preferred embodiments of the present invention will be explained with reference to accompanying drawings. The embodiments are not intended to limit the disclosed technology. The embodiments below may be combined as appropriate without containing inconsistencies.

### [a] First Embodiment

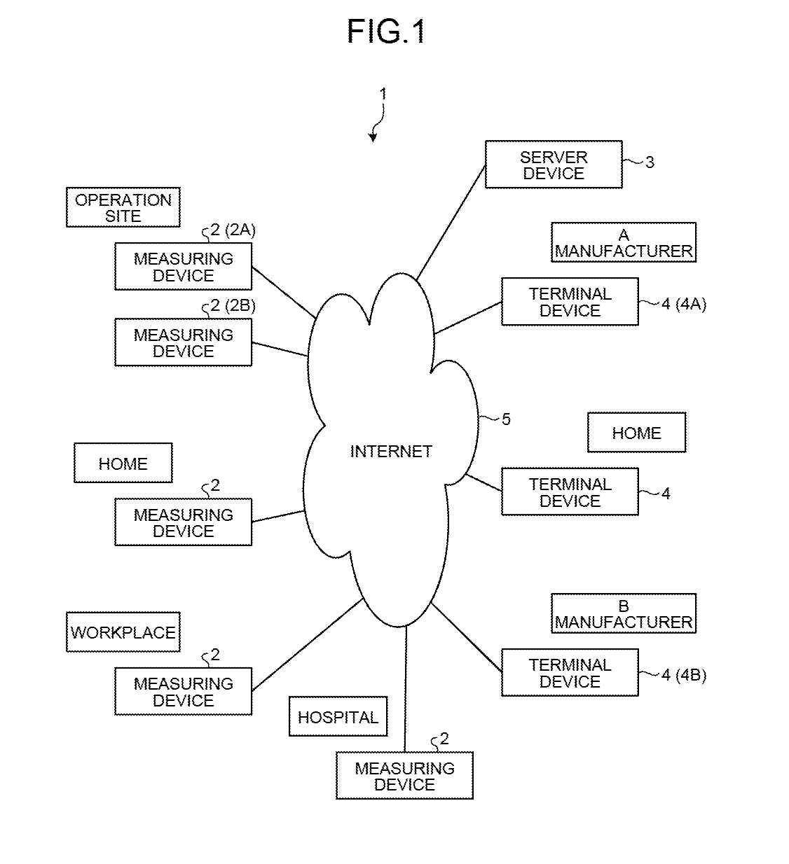

FIG, 1 is an example diagram for explaining a measuring system 1 according to an embodiment of the present invention. The measuring system 1 illustrated in FIG. 1 includes a plurality of measuring devices 2, a server device 3, and a plurality of terminal devices 4. The measuring devices 2 are arranged at homes, operation sites, workplaces, and hospitals, for example, to measure biological information on users. Examples of the measuring devices 2 include, but are not limited to, sphygmomanometers, scales, thermometers, alcohol detectors, sleep measuring instrument, etc. The measuring devices 2 are not limited to measuring devices that measure biological information and may be various sensors, such as speed sensors and rotation rate sensors of onboard drive recorders, and global positioning systems (GPSs). The server device 3 is connected to and performs communications with the measuring devices 2 via the Internet 5, for example. The server device 3 acquires measurement results from the measuring devices 2 via the Internet 5.

The terminal devices 4 are computers, for example, provided to persons and companies serving as contractors of the measuring system 1, such as users who need the measurement results of the measuring devices 2, manufacturers that manufacture the measuring devices 2, and companies that use the measurement results of the measuring devices 2. The terminal devices 4 include a terminal device 4A provided to an A manufacturer and a terminal device 4B provided to a B manufacturer, for example. The terminal devices 4 are connected to and perform communications with the server device 3 via the Internet 5, for example.

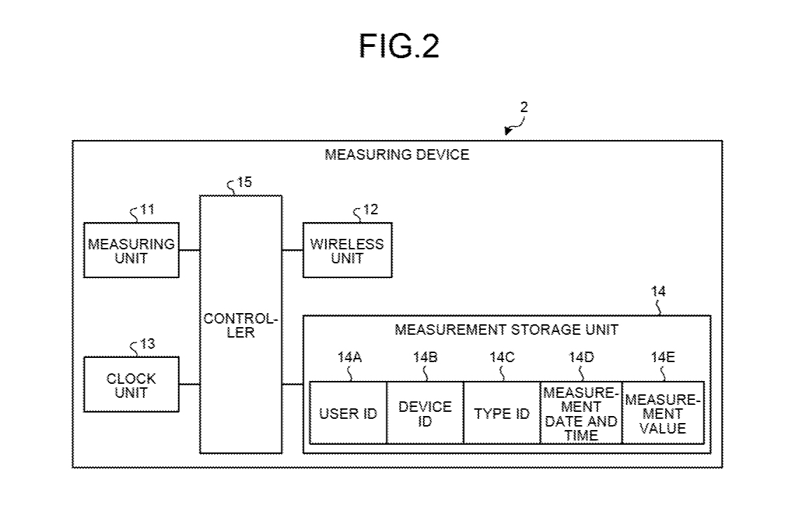

FIG. 2 is an example diagram for explaining the measuring device 2. The measuring device 2 illustrated in FIG. 2 includes a measuring unit 11, a wireless unit 12, a clock unit 13, a measurement storage unit 14, and a controller 15. In a case where the measuring device 2 is a pulsimeter, the measuring unit 11 is a wristband-shaped contact pulsimeter that measures the pulse of a user in contact with the body of the user, for example. Alternatively, the measuring unit 11 may be an ear-clip-shaped non-contact pulsimeter that measures the pulse of the user using millimeter waves or microwaves without being in contact with the body of the user, for example. In a case where the measuring device 2 is a sphygmomanometer, the measuring unit 11 is a contact or non-contact blood pressure measuring unit that measures the blood pressure of the user, for example. In a case where the measuring device 2 is a scale, the measuring unit 11 is a contact or non-contact weight measuring unit that measures the weight of the user, for example. In a case where the measuring device 2 is a thermometer, the measuring unit 11 is a contact or non-contact body temperature measuring unit that measures the body temperature of the user, for example. In a case where the measuring device 2 is a measuring device that detects a breath alcohol concentration, for example, the measuring unit 11 is a measuring unit that measures the breath alcohol concentration of the user. In a case where the measuring device 2 is a sleep measuring device, the measuring unit 11 measures the quality of sleep of the user. In a case where the measuring device 2 is a GPS that measures the present position, for example, the measuring unit 11 is a GPS measuring unit that measures the present position.

The wireless unit 12 is a communication interface that is connected to and performs communications with the Internet 5 in a wireless manner, for example. In a case where the measuring device 2 does not include the wireless unit 12, the measuring device 2 may have a function to be connected to and perform communications with the Internet 5 using a terminal device, such as a smartphone. The clock unit 13 measures the date and time of measurement performed by the measuring unit 11, for example. The measurement storage unit 14 is an area that stores therein measurement results, such as measurement values obtained on each measurement date and time, in a manner associated with respective user IDs for identifying the users of the measuring device 2.

The measurement storage unit 14 stores therein a measurement result of each user ID 14A, including a device ID 14B, a type ID 14C, a measurement date and time 14D, and a measurement value 14E. The user ID 14A is user identification information for identifying the user of the measuring device 2. The device ID 14B is device identification information for identifying the measuring device 2 of each manufacturer, for example. The device ID 14B is stored in the measurement storage unit 14. The type ID 14C is identification information for identifying the type of measurement performed by the measuring device 2, that is, the type of data, such as the pulse, the blood pressure, and the alcohol concentration. The measurement date and time 14D is a date and time of measurement performed by the measuring unit 11 measured by the clock unit 13. The measurement value 14E is a measurement value obtained by the measuring unit 11.

When the measuring unit 11 obtains a measurement value, the controller 15 stores the device ID 14B of the measuring device 2, the type ID 14C, the measurement date and time 14D, and the measurement value 14E in a manner associated with the user ID 14A for identifying the user in the measurement storage unit 14 as a measurement result.

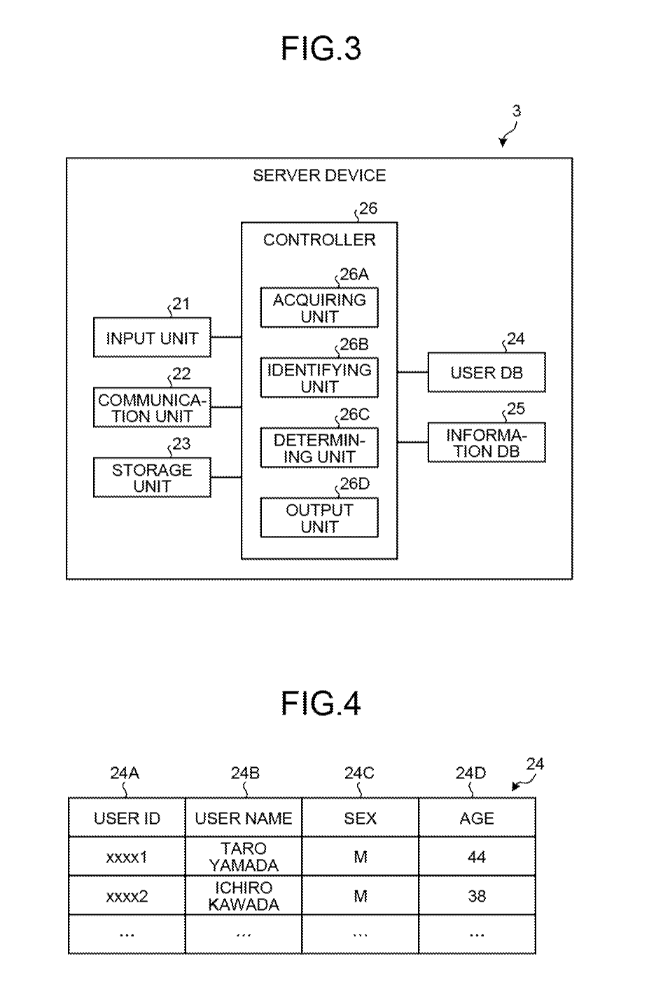

FIG. 3 is an example diagram for explaining the server device 3. The server device 3 illustrated in FIG. 3 includes an input unit 21, a communication unit 22, a storage unit 23, a user DB 24, an information DB 25, and a controller 26. The server device 3 acquires measurement results from the measuring devices 2 via the Internet 5.

The input unit 21 is an input interface that receives various commands. The communication unit 22 is a communication interface that is connected to and performs communications with the Internet 5, for example. The storage unit 23 is an area that stores therein various types of information, such as various computer programs.

The user DB 24 is an area that stores therein personal information on the users corresponding to the respective user IDs 24A for identifying the users. FIG. 4 is an example diagram for explaining a record configuration of the user DB 24. The user DB 24 illustrated in FIG. 4 is an area that stores therein personal information including a user ID 24A, a user name 24B, a sex 24C, and an age 24D in a manner associated with one another. The user ID 24A is identification information for identifying the user, for example. The user name 24B is the full name of the user, for example. The sex 24C is the sex of the user, for example. The age 24D is the age and the date of birth of the user, for example. The controller 26, for example, updates and registers the user ID 24A, the user name 24B, the sex 24C, and the age 24D in the user DB 24 by an input operation received from the terminal device 4.

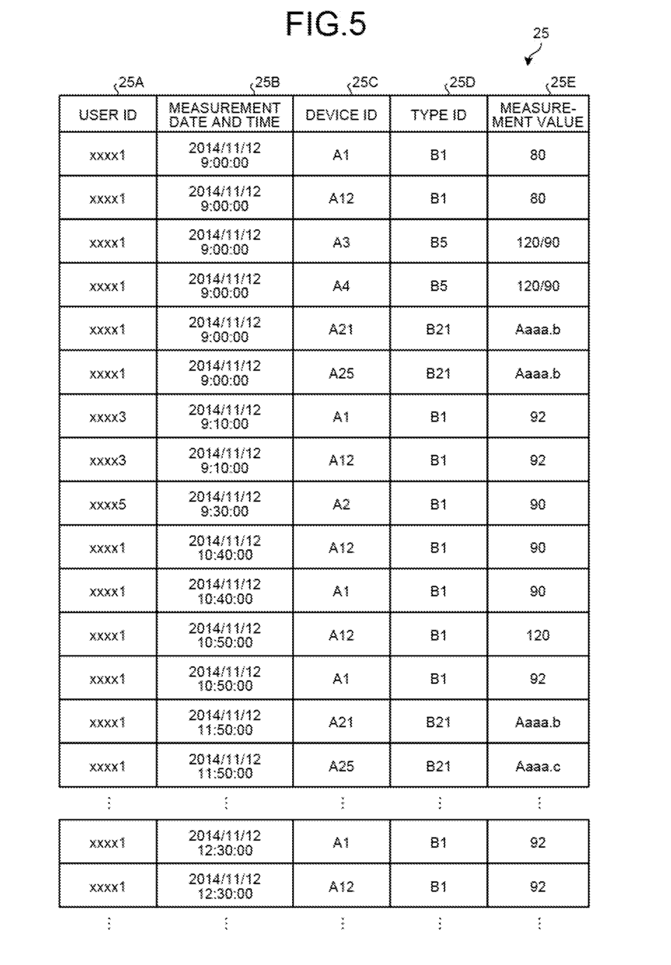

The information DB 25 is a storage area that stores therein measurement results obtained by the measuring devices 2 for each user ID 25A for identifying the user. FIG. 5 is an example diagram for explaining a record configuration of the information DB 25. The information DB 25 stores therein measurement results each including a user ID 25A, a measurement date and time 25B, a device ID 25C, a type ID 25D, and a measurement value 25E in a manner associated with one another. The user ID 25A is identification information for identifying the user, for example. The measurement date and time 25B is a date and time of measurement performed by the measuring device 2. The device ID 25C is identification information for identifying the measuring device 2 that performs the measurement. The type ID 25D is identification information for identifying the type of measurement performed by the measuring device 2 that performs the measurement. The measurement value 25E is a measurement value obtained by the measuring device 2 that performs the measurement. The type ID 25D illustrated in FIG. 5 includes “B1” indicating the blood pressure, “B5” indicating the pulse, and “B21” indicating the GPS coordinate position.

The controller 26 acquires the measurement results of the respective users from the measuring devices 2 via the communication unit 22. The controller 26 stores the user ID, the measurement date and time, the device ID, the type ID, and the measurement value in the acquired measurement result in the information DB 25 as the measurement result including the user ID 25A, the measurement date and time 25B, the device ID 25C, the type ID 25D, and the measurement value 25E, respectively.

Let us assume a case where a measuring device 2A is a contact sphygmomanometer manufactured by the A manufacturer and arranged at an operation site and where a measuring device 2B is a non-contact sphygmomanometer manufactured by the B manufacturer and arranged at the same operation site, for example. In this case, “A1” denotes the device ID 25C of the measuring device 2A, “A12” denotes the device ID 25C of the measuring device 2B, and “B1” denotes the type ID 25D of the blood pressure.

The measuring device 2A measures the blood pressure of the user by the measuring unit 11. The measuring device 2A stores a measurement result including a user ID “xxxx1”, the device ID “A1”, the type ID “B1”, the measurement value 25E, and the measurement date and time 25B in the measurement storage unit 14. The measuring device 2A transmits the measurement result stored in the measurement storage unit 14 to the server device 3 through the wireless unit 12 via the Internet 5 at a predetermined timing. The predetermined timing is a periodic timing of every predetermined period of time, a timing at which predetermined time comes, or a timing at which measurement for the measurement value is completed, for example.

Similarly, the measuring device 2B arranged at the same operation site measures the blood pressure of the user by the measuring unit 11. The measuring device 2B stores a measurement result including the user ID “xxxx1”, the device ID “A12”, the type ID “B1”, the measurement value 25E, and the measurement date and time 25B in the measurement storage unit 14. The measuring device 2B transmits the measurement result stored in the measurement storage unit 14 to the server device 3 through the wireless unit 12 via the Internet 5 at the predetermined timing.

When receiving the measurement results from the measuring devices 2, the controller 26 in the server device 3 stores the measurement results in the information DB 25. The controller 26 manages data in the user DB 24 and the information DB 25 in a manner associated with each other by the user ID 24A (25A).

Let us also assume a case where the measuring device 2A is a contact pulsimeter arranged at an operation site and where the measuring device 2B is a non-contact pulsimeter arranged at the same operation site, for example. In this case, “A3” denotes the device ID 25C of the measuring device 2A, “A4” denotes the device ID 25C of the measuring device 2B, and “B5” denotes the type ID 25D of the pulse, for example.

The measuring device 2A arranged at the operation site measures the pulse of the user by the measuring unit 11. The measuring device 2A stores a measurement result including the user ID “xxxx1”, the device ID “A3”, the type ID “B5”, the measurement value 25E, and the measurement date and time 25B in the measurement storage unit 14. The measuring device 2A transmits the measurement result stored in the measurement storage unit 14 to the server device 3 through the wireless unit 12 via the Internet 5 at the predetermined timing.

Similarly, the measuring device 2B arranged at the same operation site measures the pulse of the user by the measuring unit 11. The measuring device 2B stores a measurement result including the user ID “xxxx1”, the device ID “A4”, the type ID “B5”, the measurement value 25E, and the measurement date and time 25B in the measurement storage unit 14. The measuring device 2B transmits the measurement result stored in the measurement storage unit 14 to the server device 3 through the wireless unit 12 via the Internet 5 at the predetermined timing.

Let us also assume a case where the measuring device 2A is a GPS of a smartphone arranged at an operation site and where the measuring device 2B is a GPS mounted on a vehicle arranged at the same operation site, for example. In this case, “A21” denotes the device ID 25C of the measuring device 2A, “A25” denotes the device ID 25C of the measuring device 2B, and “B21” denotes the type ID 25D of the GPS, for example.

The measuring device 2A arranged at the operation site measures the present position coordinates by the measuring unit 11. The measuring device 2A stores a measurement result including the user ID “xxxx1”, the device ID “A21”, the type ID “B21”, the measurement value 25E, and the measurement date and time 25B in the measurement storage unit 14. The measuring device 2A transmits the measurement result stored in the measurement storage unit 14 to the server device 3 through the wireless unit 12 via the Internet 5 at the predetermined timing.

Similarly, the measuring device 2B arranged at the same operation site measures the present position coordinates by the measuring unit 11. The measuring device 2B stores a measurement result including the user ID “xxxx1”, the device ID “A25”, the type ID “B21”, the measurement value 25E, and the measurement date and time 25B in the measurement storage unit 14. The measuring device 2B transmits the measurement result stored in the measurement storage unit 14 to the server device 3 through the wireless unit 12 via the Internet 5 at the predetermined timing.

The controller 26 reads computer programs stored in the storage unit 23 and executes the read computer programs, thereby performing processes as functions. The controller 26 has a functional configuration including an acquiring unit 26A, an identifying unit 26B, a determining unit 26C, and an output unit 26D. The acquiring unit 26A acquires measurement results from the measuring devices 2 and stores the acquired measurement results in the information DB 25. The identifying unit 26B determines whether measurement results having the same user ID 25A and the same type ID 25D are present in the information DB 25 based on the acquired measurement results. If measurement results having the same user ID 25A and the same type ID 25D are present, the identifying unit 26B identifies the measurement results having the same user ID 25A and the same type ID 25D in the information DB 25. The identifying unit 26B further determines whether measurement results having different device IDs 25C are present in the identified measurement results. If measurement results having different device IDs 25C are present, the identifying unit 26B identifies the measurement results having different device IDs 25C. from the identified measurement results.

The determining unit 26C generates time-series variation data indicating time-series variation in the measurement value of each device ID 25C based on the measurement value 25E and the measurement date and time 25B in the identified measurement results. The time-series variation data indicates time-series variation in the measurement value based on the measurement value 25E and the measurement date and time 25B in the measurement results. The measurement date and time of the compared measurement values is not limited to the same overlapping period and is set for each type of measurement, that is, each type ID. The overlapping period means a period in which the measurement dates and times of the compared measurement values are the same. The determining unit 26C also determines whether the time-series variation data of the measurement values are different between the different measuring devices 2. The determining unit 26C, for example, compares the pieces of time-series variation data of the measurement values obtained on the same measurement date and time by the different measuring devices 2. The determining unit 26C determines whether a difference beyond an error range is present between the compared pieces of time-series variation data. If a difference beyond the error range is present, the determining unit 26C determines that the time-series variation data are different. The error range used to determine whether the time-series variation data are different is set in advance for each type ID. If no difference beyond the error range is present between the compared pieces of time-series variation data, the determining unit 26C determines that the time-series variation data are not different.

If the time-series variation data are different between the different measuring devices 2, the output unit 26D identifies a measuring device 2 in an abnormal state. The output unit 26D identifies a measuring device 2 in an abnormal state as follows: if the time-series variation data are different, the output unit 26D compares the measurement values with a reference measurement value; and if a measurement value is significantly different from the reference measurement value, the output unit 26D determines the measuring device 2 that outputs the measurement value to be in an abnormal state. A normal measurement value is set in advance as an initial value of the reference measurement value. The output unit 26D identifies the measuring device 2 in an abnormal state between the measuring devices 2 and outputs an alarm indicating the abnormal state of the measuring device 2 to the terminal device 4 of a predetermined destination. The alarm has warning information containing the abnormal state of the measuring device 2 and measurement results including the user ID, the device ID, the type ID, the measurement value, the measurement date and time, and the like of the measuring device 2 in the abnormal state. The predetermined destination is destination information on the terminal device 4 belonging to a contractor of the measuring system 1, such as destination information on the terminal device 4 belonging to the user of the measuring device 2 and destination information on the terminal device 4 belonging to the manufacturer of the measuring device 2.

If the server device 3 identifies measurement results having the same user ID and the same type ID in the information DB 25 in every predetermined period, and the time-series variation data of the measurement values in the identified measurement results are different between the measuring devices 2, the server device 3 identifies a measuring device 2 in an abnormal state. The server device 3 outputs an alarm indicating the abnormal state of the measuring device 2 to the terminal device 4 of a contractor. Specifically, the server device 3 refers to the information DB 25 in every predetermined period. If the time-series variation data of the measurement values associated with the same user ID and the same type ID are different between the measuring devices 2, the server device 3 identifies the measuring device 2 in an abnormal state and outputs an alarm to the terminal device 4 of the predetermined destination. In other words, the server device 3 employs a push technique.

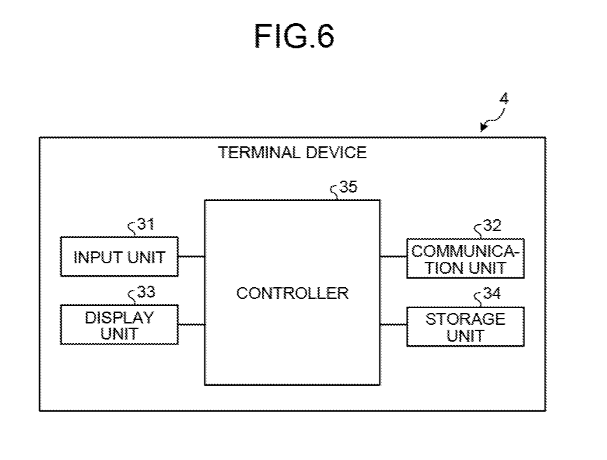

FIG. 6 is an example diagram for explaining the terminal device 4. The terminal device 4 is a terminal belonging to a contractor of the measuring system 1, such as a computer and a smartphone arranged at home of the user of the measuring device 2 and a computer belonging to the manufacturer of the measuring device 2.

The terminal device 4 illustrated in FIG. 6 includes an input unit 31, a communication unit 32, a display unit 33, a storage unit 34, and a controller 35. The input unit 31 is an input interface that receives various commands. The communication unit 32 is a communication interface that is connected to and performs communications via the Internet 5, for example. The display unit 33 is an output interface that displays-various types of information. The storage unit 34 is an area that stores therein various types of information. The controller 35 collectively controls the terminal device 4.

When receiving an alarm from the server device 3, the controller 35 in the terminal device 4 displays warning information in the alarm on a screen. Based on the warning information, the user of the terminal device 4 can recognize the abnormal state of the measuring device 2 and the user ID, the device ID, the type ID, the measurement value, and the measurement date and time of the measuring device 2 in the abnormal state.

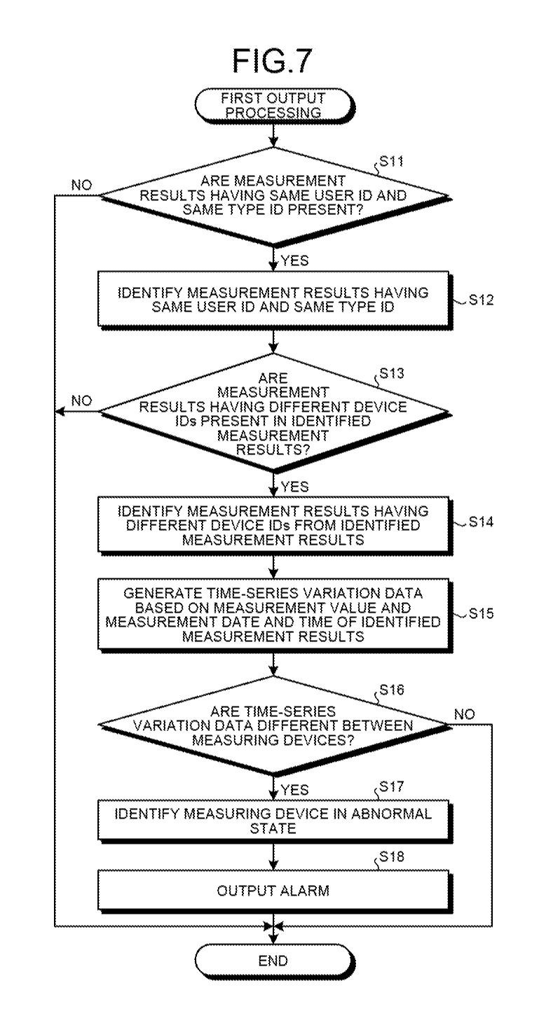

The following describes an operation performed by the measuring system 1 according to the first embodiment. FIG. 7 is an example flowchart of a processing operation performed by the server device 3 relating to first output processing. The first output processing illustrated in FIG. 7 is processing for out put ting an alarm when the time-series variation data of the measurement values are different between different measuring devices 2 in the measurement results having the same user ID and the same type ID.

In FIG. 7, the identifying unit 26B of the controller 26 in the server device 3 determines whether measurement results having the same user ID 25A and the same type ID 25D are present in the measurement results stored in the information DB 25 (Step S11). If measurement results having the same user ID 25A and the same type ID 25D are present (Yes at Step S11), the identifying unit 26B identifies the measurement results having the same user ID 25A and the same type ID 25D in the information DB 25 (Step S12). The identifying unit 26B, for example, identifies the measurement results having the user ID “xxxx1” and the type ID “B1” in the information DB 25.

The identifying unit 26B determines whether measurement results having different device IDs 25C are present in the identified measurement results (Step S13). If measurement results having different device IDs 25C are present (Yes at Step S13), the identifying unit 26B identifies the measurement results having different device IDs 25C from the measurement results identified at Step S12 (Step S14). The identifying unit 26B, for example, identifies the measurement results having the device IDs “A1” and “A12” from the measurement results having the user ID “xxxx1” and the type ID “B1” in the information DB 25.

The determining unit 26C in the controller 26 generates pieces of time-series variation data of the respective device IDs 25C based on the measurement value 25E and the measurement date and time 25B in the measurement results having the respective device IDs 25C identified at Step S14 (Step SIS). Specifically, the determining unit 26C generates pieces of time-series variation data of the respective device IDs “A1” and “A12” from the measurement results having the user ID “xxxx1” and the type ID “B1”. The determining unit 26C determines whether the pieces of time-series variation data are different between the measuring devices 2 (Step S16). If the time-series variation data are different between the measuring devices 2 (Yes at Step S16), the output unit 26D in the controller 26 identifies a measuring device 2 in an abnormal state (Step S17). After identifying the measuring device 2 in an abnormal state between the measuring devices 2, the output unit 26D outputs an alarm indicating the abnormal state of the measuring device 2 to the terminal device 4 of a predetermined destination (Step S18) and finishes the processing operation illustrated in FIG. 7. The predetermined destination is destination information on the terminal device 4 belonging to the manufacturer of the measuring device 2, for example. In a case where the output unit 26D detects an abnormal state of the measuring device 2A, for example, the output unit 26D outputs an alarm to the terminal device 4 belonging to the A manufacturer of the measuring device 2A.

If no measurement result having the same user ID and the same type ID is present (No at Step S11) or if no measurement result having different device IDs is present (No at Step S13), the identifying unit 26B finishes the processing operation illustrated in FIG. 7. If the generated time-series variation data are not different between the measuring devices 2 (No at Step S16), the determining unit 26C determines that the measuring devices 2 are in a normal state and finishes the processing operation illustrated in FIG. 7.

The server device 3 identifies measurement results having different device IDs from the measurement results having the same user ID and the same type ID in the information DB 25. If the time-series variation data are different between the measuring devices 2 in the identified measurement results, the server device 3 identifies a measuring device 2 in an abnormal state. After identifying the measuring device 2 in an abnormal state, the server device 3 outputs an alarm to the terminal device 4 of a predetermined destination. Based on the alarm, the user of the terminal device 4 can recognize the abnormal state of the measuring device 2 and the device ID, the user ID, the type ID, the measurement value, and the measurement date and time of the measuring device 2.

In a case where the user of the terminal device 4 is the manufacturer of the measuring device 2, the manufacturer can recognize the abnormal state of the measuring device 2 based on the alarm. In a case where the user of the terminal device 4 is the user of the measuring device 2, the user can recognize the abnormal state of the measuring device 2 based on the alarm.

The server device 3 compares the pieces of time-series variation data of the measurement values associated with the same user ID and the same type ID obtained on the same date and time. As a result, the server device 3 can detect an abnormal state of the measuring device 2 on the same date and time.

If the time-series variation data are different between the measuring devices 2, the server device 3 according to the first embodiment identifies a measuring device 2 in an abnormal state and outputs an alarm to the terminal device 4 belonging to the manufacturer or the like of the measuring device 2 in an abnormal state. If the time-series variation data are different between the measuring devices 2, however, the server device 3 may output an alarm to the terminal devices 4 belonging to the manufacturers of the respective measuring devices 2 and the terminal devices 4 belonging to the users thereof without identifying the measuring device 2 in an abnormal state.

While the server device 3 according to the first embodiment outputs an alarm including the abnormal state and the measurement results of the measuring device 2 to the terminal device 4 of the predetermined destination when the time-series variation data are different, the embodiment is not limited thereto. The alarm may include the amount of difference between the measurement values of the different measuring devices 2.

While the type ID according to the first embodiment indicates the blood pressure or the pulse, for example, the type ID is not limited to these. The type ID may be various types of information, including various types of biological information, such as vital signs of the alcohol concentration, the weight, the height, and the percent of body fat, or information on the present position of a GPS, for example, and can be changed as appropriate. In the case of a GPS, the server device 3 performs the following processing: if the time-series variation data of the measurement values are different between different GPSs, the server device 3 identifies a GPS in an abnormal state and outputs an alarm to the terminal devices 4 belonging to the manufacturer of the GPS in an abnormal state and belonging to the user thereof.

While the determining unit 26C determines that the pieces of time-series variation data are different when a difference beyond the error range is present between the compared pieces of time-series variation data, the embodiment is not limited thereto. The determining unit 26C may determine that the pieces of time-series variation data are different when an error is present between them without setting any error range.

While the predetermined destination to which the output unit 26D outputs an alarm is the terminal device 4 of the manufacturer or the terminal device 4 of the user who is a contractor, for example, the predetermined destination is not limited to this. The predetermined destination, for example, may be the terminal device 4 of a contractor who needs measurement results relating to the type ID corresponding to the time-series variation data, for example. Alternatively, the predetermined destination may be the terminal device 4 of a contractor who needs measurement results relating to the device ID corresponding to the time-series variation data or the terminal device 4 of a contractor who needs measurement results relating to the user ID corresponding to the time-series variation data.

As described above, the server device 3 refers to the information DB 25 in every predetermined period. If the time-series variation data are different between the measuring devices 2 in the measurement results having the same user ID and the same type ID, the server device 3 identifies the measuring device 2 in an abnormal state and outputs an alarm to the terminal device 4 of the predetermined destination. In other words, the server device 3 employs a push technique. The embodiment, however, is not limited thereto, and the server device 3 may employ a pull technique. In the pull technique, the server device 3 refers to the information DB 25 in response to a request from the terminal device 4 of a contractor. If the time-series variation data are different between the measuring devices 2 in the measurement results having the same user ID and the same type ID, the server device 3 outputs an alarm to the terminal device 4 serving as the source of the request.

While the server device 3 compares the pieces of time-series variation data obtained on the same date and time between the measuring devices 2, the server device 3 may compare pieces of time-series variation data of the measurement values obtained at the same time on different dates. Alternatively, the server device 3 may compare pieces of time-series variation data of the measurement values obtained on different dates and times. Still alternatively, the server device 3 may compare pieces of time-series variation data of the averages of the measurement values obtained by the respective measuring devices 2 on a monthly or a daily basis.

While the output unit 26D uses a reference measurement value to determine whether the measuring device 2 is in an abnormal state, the embodiment is not limited thereto. In a case where the measuring device 2 is a sphygmomanometer, for example, the output unit 26D may sequentially update the measurement value of the user to use the average measurement value of the user as the reference measurement value. As a result, the output unit 26D can provide the reference measurement value suitable for the user to determine whether the measuring device 2 is in an abnormal state.

The first embodiment generates the time-series variation data based on the measurement value 25E and the measurement date and time 25B associated with the same user ID and the same type ID obtained by two measuring devices 2. If the time-series variation data are different between the measuring devices 2, the first embodiment identifies a measuring device 2 in an abnormal state and outputs an alarm indicating the abnormal state of the measuring device 2. The number of measuring devices 2, however, is not limited to two. The server device 3 may generate three or more pieces of time-series variation data based on the measurement value 25E and the measurement date and time 25B associated with the same user ID and the same type ID obtained by three or more measuring devices 2. If the generated three or more pieces of time-series variation data are different among the measuring devices 2, the server device 3 identifies a measuring device 2 in an abnormal state and outputs an alarm indicating the abnormal state of the measuring device 2.

If the time-series variation data of the measurement values are different between the measuring devices 2 in the measurement results having the same user ID and the same type ID, the first embodiment identifies a measuring device 2 in an abnormal state and outputs an alarm indicating the abnormal state of the measuring device 2. The embodiment, however, is not limited thereto. The embodiment may identify, in the information DB 25, measurement results having the type ID specified by search conditions included in the contract of each contractor and output the identified measurement results to the terminal device 4 of the contractor.

The server device 3 identifies measurement results having the same user ID and the same type ID in the information DB 25, identifies measurement results having different device IDs from the identified measurement results, and generates the time-series variation data based on the measurement date and time and the measurement value in the identified measurement results. The embodiment, however, is not limited thereto, and the server device 3 may generate and output the time-series variation data independently of the user ID. In other words, the server device 3, for example, may identify measurement results having a specific type ID in the information DB 25, identify measurement results having different device IDs from the identified measurement results, and generate and output the time-series variation data based on the identified measurement results. As described above, the method for identifying the measurement results can be changed as appropriate.

The first embodiment is not always applied to the measuring device 2 that measures biological information and may be applied to a measuring device that measures the atmospheric temperature, for example. In this case, the user ID corresponds to a place ID indicating the place at which the measuring device is arranged, the type ID indicates the atmospheric temperature, and the device ID indicates a temperature measuring device. In a case where a plurality of temperature measuring devices are arranged at the same place, the server device 3 determines whether the pieces of time-series variation data of the respective temperature measuring devices are different. If the time-series variation data are different, the server device 3 identifies a temperature measuring device in an abnormal state. The server device 3 then outputs an alarm indicating the abnormal state of the temperature measuring device to the terminal device 4 belonging to the manufacturer of the temperature measuring device in the abnormal state.

The measuring system 1 according to the first embodiment enables the server device 3 and the terminal device 4 or the like to be connected to and perform communications with each other via the Internet 5. The measuring system 1 may use a local area network (LAN) instead of the Internet 5, for example, and the network can be changed as appropriate.

While the server device 3 according to the first embodiment is a computer, and the terminal device 4 is a computer of the user, the embodiment is not limited thereto. Various functions and information of the server device 3 and the terminal device 4 may be provided by cloud computing.

While the first embodiment identifies measurement results in the information DB 25 under the search conditions of the same user ID and the same type ID, the embodiment is not limited thereto. The search conditions may include a specified time range besides the same user ID and the same type ID. The following describes the embodiment as a second embodiment of the present invention.

### [b] Second Embodiment

The controller 26 in the server device 3 stores a specified time range in the storage unit 23 as the search conditions. The specified time range can be changed as appropriate by an input operation received from the input unit 21 and an input operation received from the terminal device 4.

The identifying unit 26B identifies measurement results having the same user ID and the same type ID in the information DB 25 and identifies measurement results having different device IDs in the identified measurement results. The identifying unit 26B further identifies measurement results the measurement date and time of which falls within the specified time range from the measurement results having the different device IDs in the information DB 25.

The determining unit 26C compares the pieces of time-series variation data of the measurement values in the identified measurement results the measurement date and time of which falls within the specified time range and determines whether the time-series variation data are different. If the time-series variation data are different, the output unit 26D identifies a measuring device 2 in an abnormal state and outputs an alarm to the terminal device 4 of the predetermined destination.

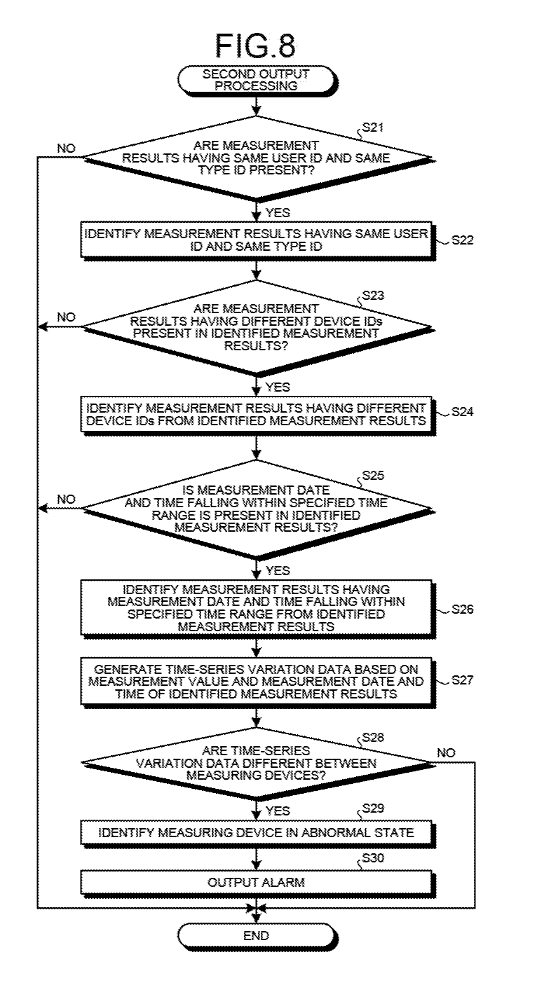

FIG. 8 is an example flowchart of a processing operation performed by the server device 3 relating to second output processing according to the second embodiment. The second output processing illustrated in FIG. 8 is processing for outputting an alarm when the time-series variation data are different in the measurement results having the same user ID and the same type ID and the measurement date and time of which falls within the specified time range.

In FIG. 8, the identifying unit 26B of the controller 26 in the server device 3 determines whether measurement results having the same user ID 25A and the same type ID 25D are present in the measurement results stored in the information DB 25 (Step S21). If measurement results having the same user ID 25A and the same type ID 25D are present (Yes at Step S21), the identifying unit 26B identifies the measurement results having the same user ID 25A and the same type ID 25D in the information DB 25 (Step S22).

The identifying unit 26B determines whether measurement results having different device IDs 25C are present in the identified measurement results (Step S23). If measurement results having different device IDs 25C are present (Yes at Step S23), the identifying unit 26B identifies the measurement results having different device IDs 25C from the measurement results identified at Step S22 (Step S24). The identifying unit 26B, for example, identifies the measurement results having the device IDs “A1” and “A12” from the measurement results having the user ID “xxxx1” and the type ID “B1” in the information DB 25.

The identifying unit 26B determines whether the measurement date and time 25B falling within the specified time range is present in the identified measurement results (Step S25). If the measurement date and time 25B falling within the specified time range is present in the identified measurement results (Yes at Step S25), the identifying unit 26B identifies measurement results having the measurement date and time 25B falling within the specified time range from the identified measurement results (Step S26).

The determining unit 26C generates pieces of time-series variation data of the respective device IDs 25C based on the measurement value 25E and the measurement date and time 25B in the measurement results having the respective device IDs 25C identified at Step S26 (Step S27), Specifically, the determining unit 26C generates pieces of time-series variation data of the respective device IDs “A1” and “A12” from the measurement results having the user ID “xxxx1” and the type ID “B1”. The determining unit 26C determines whether the generated pieces of time-series variation data are different between the measuring devices 2 (Step S28). If the time-series variation data are different between the measuring devices 2 (Yes at Step S28), the output unit 26D identifies a measuring device 2 in an abnormal state (Step S23). After identifying the measuring device 2 in an abnormal state, the output unit 26D outputs an alarm indicating the abnormal state of the measuring device 2 to the terminal device 4 of a predetermined destination (Step S30) and finishes the processing operation illustrated in FIG. 8.

If no measurement result having the same user ID and the same type ID is present (No at Step S21) or if no measurement result having different device IDs is present (No at Step S23), the identifying unit 26B finishes the processing operation illustrated in FIG. 8. If no measurement date and time 25B falling within the specified time range is present in the identified measurement results (No at Step S25), the determining unit 26C finishes the processing operation illustrated in FIG. 8. If the time-series variation data are not different between the measuring devices 2 (No at Step S28), the determining unit 26C determines that the measuring devices 2 are in a normal state and finishes the processing operation illustrated in FIG. 8.

The controller 26 identifies measurement results having different device IDs from the measurement results having the same user ID and the same type ID and the measurement date and time of which falls within the specified time range in the information DB 25. If the time-series variation data are different between the measuring devices 2 in the identified measurement results, the controller 26 identifies a measuring device 2 in an abnormal state and outputs an alarm indicating the abnormal state of the measuring device 2. As a result, the server device 3 can identify the measurement results the measurement date and time of which falls within the specified time range from the measurement results having the same user ID and the same type ID. Based on the alarm, the user of the terminal device 4 can recognize the abnormal state of the measuring device 2 on the measurement date and time falling within the specified time range between the measuring devices 2 being used and the device ID, the user ID, the type ID, the measurement value, and the measurement date and time of the measuring device 2 in the abnorma1 state.

The components of the units illustrated in the drawings are not necessarily physically configured as illustrated. In other words, the specific aspects of distribution and integration of the units are not limited to those illustrated in the drawings. All or a part of the components may be distributed or integrated functionally or physically in desired units depending on various types of loads and usage, for example.

All or a desired part of various processing functions performed by the computers serving as the server device 3 and the terminal device 4 may be carried out by a central processing unit (CPU) (or a microcomputer, such as a micro processing unit (MPU) and a micro controller unit (MCU) ). Needless to say, all or a desired part of the various processing functions may be provided by a computer program analyzed and executed by the CPU (or a microcomputer, such as an MPO and an MCU) or hardware by-wired logic.

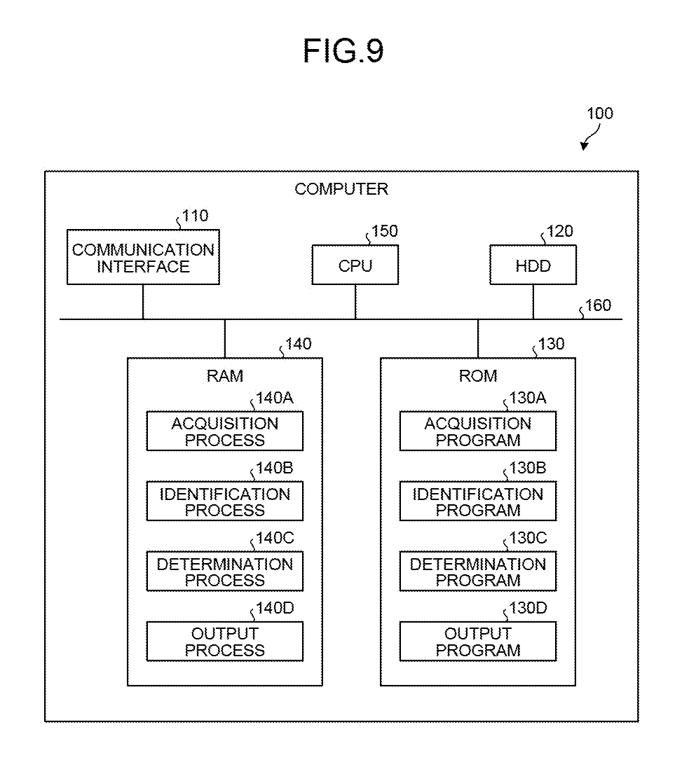

The various types of processing described in the embodiments above can be performed by a computer executing a computer program prepared in advance. The following describes an example of the computer that executes a computer program having the same functions as those according to the embodiments above. FIG. 9 is an example diagram for explaining a computer 100 that executes an output program.

As illustrated in FIG. 9, the computer 100 that executes the output program includes a communication interface 110, a hard disk drive (HDD) 120, a read only memory (ROM) 130, a random access memory (RAM) 140, a CPU 150, and a bus 160.

The ROM 130 stores therein in advance an output program for carrying out the same functions as those according to the embodiments above. Processing programs may be stored not in the ROM 130 but in a recording medium that can be read by a drive, which is not illustrated. Examples of the recording medium include, but are not limited to, a portable recording medium, such as a compact disc read only memory (CD-ROM), a digital versatile disc (DVD), a universal serial bus (USB) memory, and an SD card, a semiconductor memory, such as an HDD and a flash memory, etc. The processing programs are an acquisition program 130A, an identification program 130B, a determination program 130C, and an output program 130D. The acquisition program 130A, the identification program 130B, the determination program 130C, and the output program 130D may be integrated or distributed as appropriate.

The CPU 150 reads the acquisition program 130A, the identification program 130B, the determination program 130C, and the output program 130D from the ROM 130 and executes the read programs. The CPU 150 causes the programs 130A, 130B, 130C, and 130D to function as an acquisition process 140A, an identification process 140B, a determination process 140C, and an output process 140D, respectively, on the RAM 140.

The CPU 150 acquires information including user identification information, device identification information for identifying a measuring device, a measurement value of each measurement type obtained by the measuring device, and measurement time of the measurement-value from different measuring devices. The CPU 150 identifies information including measurement values associated with the same user identification information and the same measurement type from the acquired information. The CPU 150 determines whether temporal variation in the measurement values included in the identified information is different between a first measuring device and a second measuring device identified by the device identification information included in the identified information. If the temporal variation in the measurement values is different between the first measuring device and the second measuring device, the CPU 150 outputs the determination result. As a result, the CPU 150 can output an abnormal state of a measuring device.

According to an aspect, an output device can output an abnormal state of a measuring device.

All examples and conditional language recited herein are intended for pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although the embodiments of the present invention have been described in detail, it should be understood that the various changes, substitutions, and alterations could be made hereto without departing from the spirit and scope of the invention.

What is claimed is:

1. An output device comprising a processor, wherein the processor executes a process comprising:
acquiring information including user identification information, device identification information for identifying a measuring device, a measurement value of each measurement type obtained by the measuring device, and measurement time of the measurement value, from different measuring devices;
identifying information including measurement values associated with the same user identification information and the same measurement type, from the acquired information;
determining whether time-series variation in the measurement values included in the identified information is different between a first measuring device and a second measuring device identified by the device identification information included in the identified information; and
outputting a result of difference when the time-series variation in the measurement values is different between the first measuring device and the second measuring device.

2. The output device according to claim 1, wherein the time-series variation is temporal variation in a period when the measurement times are the same or temporal variation in a period when the measurement times are the same on a daily basis or a monthly basis.

3. The output device according to claim 1, wherein the outputting includes transmitting the result to a destination corresponding to the first measuring device or an access source in response to a request from the access source.

4. The output device according to claim 1, wherein the outputting includes outputting information indicating that the measurement value obtained by the first measuring device is different from the measurement value obtained by the second measuring device in the identified information.

5. The output device according to claim 1, wherein the outputting includes outputting information indicating an amount of difference between the measurement value obtained by the first measuring device and the measurement value obtained by the second measuring device in the identified information.

6. An output device comprising a processor, wherein the processor executes a process comprising:
acquiring information including user identification information, device identification information for identifying a measuring device, a measurement value of each measurement type obtained by the measuring device, and measurement time of the measurement value, from different measuring devices;
identifying information including measurement values associated with the same user identification information and the same measurement type and measurement time failing within a specified time range, from the acquired information;
determining whether time-series variation in the measurement values included in the identified information is different between a first measuring device and a second measuring device identified by the device identification information included in the identified information; and
outputting a result of difference when the time-series variation in the measurement values is different between the first measuring device and the second measuring device.

7. An output device comprising a processor, wherein the processor executes a process comprising:
acquiring information including user identification information, device identification information for identifying a measuring device, a measurement value of each measurement type obtained by the measuring device, and measurement time of the measurement value, from different measuring devices;
identifying information including measurement values associated with the same user identification information and the same measurement type, from the acquired informati on;
determining whether time-series variation in the measurement values included in the identified information is different among at least a first measuring device, a second measuring device, and a third measuring device identified by the device identification information included in the identified information; and
outputting a result of difference when the time-series variation in the measurement values is different among the first measuring device, the second measuring device, and the third measuring device,

8. An output method comprising:
acquiring, by a processor, information including user identification information, device identification information for identifying a measuring device, a measurement value of each measurement type obtained by the measuring device, and measurement time of the measurement value, from different measuring devices;
identifying, by the processor, information including measurement values associated with the same user identification information and the same measurement type, from the acquired information;
determining, by the processor, whether temporal variation in the measurement values included in the identified information is different between a first measuring device and a second measuring device identified by the device identification information included in the identified information; and
outputting, by the processor, a result of the determination when the temporal variation in the measurement values is different between the first measuring device and the second measuring device.

9. A non-transitory computer-readable recording medium having stored therein an output program that causes a computer to execute a process comprising:
acquiring information including user identification information, device identification information for identifying a measuring device, a measurement value of each measurement type obtained by the measuring device, and measurement time of the measurement value, from different measuring devices;
identifying information including measurement values associated with the same user identification information and the same measurement type, from the acquired information;
determining whether temporal variation in the measurement values included in the identified information is different between a first measuring device and a second measuring device identified by the device identification information included in the identified information; and
outputting a result of the determination when the temporal variation in the measurement values is different between the first measuring device and the second measuring device.
